(19) United States
(12) Patent Application Publication
(10) Pub. No.: US 2025/0389742 A1
(43) Pub. Date: Dec. 25, 2025

(54) A SYSTEM ARRANGED TO MONITOR AND CONTROL APPLICATION OF A MILK SAMPLE TO A LATERAL FLOW STICK

(71) Applicant: DELAVAL HOLDING AB, Tumba (SE)

(72) Inventors: Kristjan Freyr GUDMUNDSSON, Tumba (SE); Lars MOLNIT, Tumba (SE)

(21) Appl. No.: 18/879,736

(22) PCT Filed: Jun. 21, 2023
(86) PCT No.: PCT/SE2023/050642

§ 371 Date: Dec. 27, 2024

(30) Foreign Application Priority Data
Jun. 29, 2022 (SE) 2250806-3

(51) Int. Cl. G01N 35/10; B01L 3/00; G01N 33/04; G01N 35/00

(52) U.S. Cl. CPC G01N 35/1011; B01L 3/5023; G01N 33/04; G01N 35/00009; B01L 2300/069; B01L 2300/0825

(57) ABSTRACT

A system for monitoring and controlling application of milk samples to lateral flow test sticks. The system includes a milk dosing assembly with a needle that moves between an application position contacting a test stick and a retracted position. A moveable structure holds and positions the dosing assembly, while a drive unit controls the movement. A controller determines and maintains proper spacing between the needle tip and test stick by comparing measured distances with predetermined values and making adjustments as needed. The system enables accurate and automated application of milk samples for biomarker testing while preventing sample cross-contamination and ensuring proper sample delivery to the test area.

### TECHNICAL FIELD

This document discloses a system according to claim 1. More particularly, a system is described, arranged to monitor and control application of a milk sample extracted from an animal to a sample pad of a lateral flow stick, wherein the lateral flow stick is arranged to indicate presence of a biomarker in the milk sample.

### BACKGROUND

On an animal farm, it is important to keep the animals healthy in order to enhance milk/meat production. For example, it is important to inseminate animals at an optimal moment in order to successfully fertilise the animal, such as a cow. In case the animal is not successfully inseminated, milk production is affected.

Several biomarker measurements may be made on the animal, such as e.g. measuring levels of progesterone, LDH (Lactate Dehydrogenase), BHB (Beta-Hydroxybutyrat) and urea. Thereby important information concerning e.g. heat detection and/or pregnancy of the individual animal may be made (based on measured progesterone level), as well as mastitis (based on LDH) and ketosis (based on BHB). Also, the energy balance may be estimated (based on urea).

Biomarker measurements may be made at the farm by applying a milk sample of the tested animal during the milking session and apply the sample on prepared lateral flow stick/dry stick. This may preferably be made in an automatised manner for example during a milking session. A milk analysis apparatus/service module may then extract a milk sample of an animal.

A known concept is presented in document WO 2018236271, in order to help the farmer in handling the lateral flow sticks in a convenient and automatised manner is to keep them mounted on a carrier tape, which in turn may be kept in a cassette. When a new milk sample is to be applied to a lateral flow stick, the carrier tape may be forwarded to the next unused lateral flow stick.

The milk sample may be provided to the lateral flow stick via a needle, which is hold in a moveable structure in the milk analysis apparatus/service module. The needle may be hold in a retracted position when the lateral flow stick, or the carrier tape comprising the lateral flow stick, is moved into position at a distance from the needle. The reason for keeping the needle in the retracted position during positioning of the lateral flow stick is to avoid that the carrier tape/lateral flow stick is harmed by the needle.

When the milk sample is to be applied, the needle tip may approach the sample pad of the lateral flow stick into an application position. The milk sample is then applied to the sample pad.

A problem that occurs in this situation is that the distance between the sample pad of the lateral flow stick and the tip of the needle may not always be the same when it is hold in its retracted position. The reasons may for example be insufficient precision during calibration of the needle and/or variations in tension in the carrier tape during use.

Thus, to move the needle the same distance from the retracted position towards the lateral flow stick may result in the needle tip not quite reaching the surface of the sample pad of the lateral flow stick which may result in milk sample residues on the outer side of the needle and as a consequence cause carry over, i.e. a milk sample of a previous animal influences a milk sample of another animal to be tested. Alternatively, the needle tip may puncture and pass the lateral flow stick and waste the milk sample outside the backside of the lateral flow stick. In both cases, the result may be a wasted milk sample which cannot be used. Possibly, also cleaning has to be made to avoid carry over between milk samples, which delay the testing capacity.

It would be desired to find a way to assist the farmer in assuring correct application of an extracted milk sample to lateral flow sticks in order to perform biomarker measurements at the farm during milking.

### SUMMARY

It is therefore an object of this invention to solve at least some of the above problems and assure correct application of an extracted milk sample to a lateral flow stick.

According to a first aspect of the invention, this objective is achieved by a system. The system is arranged to monitor and control application of a milk sample extracted from an animal to a sample pad of a lateral flow stick. The lateral flow stick is arranged to indicate presence of a biomarker in a milk sample extracted from an animal.

A biomarker, or biological marker, generally refers to a measurable indicator of some biological state or condition of the animal. The biomarker value measurement may be associated with pregnancy/reproduction of the animal.

The system comprises a milk dosing arrangement. The milk dosing arrangement comprises a tube element arranged to obtain the milk sample. Also, the milk dosing arrangement comprises a needle arranged to receive the milk sample via the tube element and apply the milk sample to a sample pad of the lateral flow stick when the milk dosing arrangement is hold in an application position. In the application position, the needle tip abuts the sample pad of the lateral flow stick.

The expression “the needle tip abuts the sample pad” means that the needle tip of the needle may abut a surface of the sample pad of the lateral flow stick, i.e. merely touching the surface of the sample pad, in the application position. Also, or alternatively, the needle tip of the needle may abut the sample pad of the lateral flow stick, wherein the surface of the sample pad is allowed to deflect in the movement direction of the needle, in the application position. Also, or alternatively, the needle tip of the needle may abut the sample pad of the lateral flow stick by penetrating the surface of the sample pad, in the application position, while not being allowed to pass the sample pad and exit on the opposite side.

In all the above-defined definitions, the problem of milk sample residues on the outer side of the needle is solved, as capillar forces of the sample pad, possibly in conjunction with gravity is allowed to act on the milk sample in order to transfer the milk sample from the needle to the sample pad of the lateral flow stick.

The system comprises a moveable structure. The moveable structure is arranged to hold the milk dosing arrangement and to move the needle of the milk dosing arrangement between the application position and a retracted position. In the application position, the needle tip of the needle abuts the sample pad of the lateral flow stick. The milk sample is applied to the sample pad via the needle, in the application position. In the retracted position, the needle tip is situated at a distance from the lateral flow stick.

The system also comprises a drive unit arranged to act on the moveable structure, thereby causing the moveable structure to move the needle of the milk dosing arrangement a predetermined distance between the retracted position and the application position.

In addition, the system comprises a controller. The controller is communicatively connected to the drive unit. The controller is configured to determine the distance between the needle tip of the needle of the milk dosing arrangement when the needle is situated in the retracted position, and the lateral flow stick. The controller is also configured to compare the determined distance with the predetermined distance. When the determined distance is differentfrom the predetermined distance, the controller is configured to update the predetermined distance to correspond with the determined distance; or adjust the distance between the needle tip of the needle of the milk dosing arrangement when the needle is situated in the retracted position and the lateral flow stick to correspond with the predetermined distance.

The predetermined distance may be stored in and retrieved from a memory device, communicatively connected with the controller.

Thanks to the provided solution, it is assured that the predetermined distance that the moveable structure is moving the needle between the retracted position and the application position corresponds with the actually measured distance between the retracted position and the application position, either by setting the predetermined distance to the determined distance, or to adjust the needle position so that the actual distance coincide with the predetermined distance.

The described solution could conveniently be made without involving manual interaction and before experiencing any failed application of milk sample when performing a lateral flow measurement. Reliable functionality of biomarker measurements of milk samples is thereby assured.

In a first implementation of the system according to the first aspect, the needle tip of the needle of the milk dosing arrangement abuts a surface of the sample pad of the lateral flow stick when the needle of the milk dosing arrangement is situated in the application position.

In a second implementation of the system according to the first aspect, or any implementation thereof, the controller may be configured to determine the distance by setting a speed of the drive unit below a speed threshold limit. Also, the controller may be configured to cause the drive unit to act on the moveable structure, thereby causing the moveable structure to move the milk dosing arrangement from the retracted position towards the lateral flow stick. In addition the controller may be configured to count each step moved by the drive unit until detecting that the needle tip of the needle abuts the sample pad of the lateral flow stick. The controller may also be configured to determine the distance based on the counted number of steps moved by the drive unit.

An advantage by basing the distance determination on the number of steps of the drive unit is that no dedicated sensor is required for determining the distance, which saves costs.

In a third implementation of the system according to the first aspect, or any implementation thereof, the controller is configured to determine the distance between the needle tip of the needle of the milk dosing arrangement when the needle is situated in the retracted position, and the lateral flow stick by setting a motor power of the drive unit below a power threshold limit, and detecting that the needle tip of the needle abuts the sample pad of the lateral flow stick by detecting that the drive unit is stalling.

In a fourth implementation of the system according to the first aspect, or any implementation thereof, the system may comprise a camera directed to capture an image of the lateral flow stick. The controller may also be configured to directly or indirectly determine the distance by causing the moveable structure to move the milk dosing arrangement from the retracted position towards the lateral flow stick. Also, the controller may be configured to cause the camera to capture at least one image of the lateral flow stick. The controller may be configured to obtain the captured image from the camera, and then detect based on image analysis of the captured image, that the needle tip of the needle abuts the sample pad of the lateral flow stick.

In a fifth implementation of the system according to the fourth implementation, the controller may be configured to detect that the needle tip of the needle abuts the sample pad of the lateral flow stick by detecting light reflections created by the needle tip when the needle tip abuts a foil covering the sample pad of the lateral flow stick.

An advantage with the fifth implementation is that any appropriate kind of drive unit could be used for moving the moveable structure. Another advantage is that a verification of the distance could be made, in combination with the methodology involving counting the number of steps made by a stepper motor according to other implementations, thereby eliminating or at least reducing risks of incorrect determination of the distance.

In a sixth implementation of the system according to the first aspect, or any implementation thereof, the device may comprise a sensor, communicatively connected to the controller. The controller may be configured to directly or indirectly determine the distance by emitting, via an emitting element of the sensor, an electromagnetic wave towards the lateral flow stick. The controller may also be configured to receive, via a receiving element of the sensor, a reflection of the emitted electromagnetic wave reflected by the lateral flow stick. In addition, the controller may be configured to measure a time period between the emission of the electromagnetic wave and the reception of the reflection of the electromagnetic wave. Also, the controller may be configured to determine the distance, directly or indirectly based on the measured time period.

In a seventh implementation of the system according to the first aspect, or any implementation thereof, the controller may be configured to determine the distance at a regular time interval.

By regular determination of the distance between the needle tip and the lateral flow stick when the needle is situated in the retracted position, any risks of failure during application of the milk sample to the sample pad of the lateral flow stick due to incorrect positioning of the needle during application of the milk sample to the sample pad is eliminated, or at least reduced.

In an eighth implementation of the system according to the first aspect, or any implementation thereof, the lateral flow stick may be arranged on a carrier comprising a plurality of lateral flow sticks. The carrier may be replaceable. The controller may be configured to detect a replacement of carriers. Also, the controller may be configured to determine the distance between the needle tip of the needle of the milk dosing arrangement when the needle is situated in the retracted position, and the lateral flow stick, upon detection of the change of carriers.

By determining the distance between the needle tip and the lateral flow stick when the needle is situated in the retracted position each time the carrier is replaced, any risks of failure during application of the milk/liquid sample to the sample pad of the lateral flow stick due to incorrect positioning of the needle during application of the milk sample to the sample pad is eliminated, or at least reduced.

In a ninth implementation of the system according to the first aspect, or any implementation thereof, the system may comprise a user interface, enabling an operator to trigger the distance determination. The controller may be configured to determine the distance when receiving a trigger signal from the operator via the user interface.

The operator may for example visually detect a deviation from a normal/expected situation concerning the position of the needle of the milk dosing arrangement when situated in the retracted position, the position of the lateral flow stick and/or the moveable structure. By determine the distance upon request by the operator, risks of failure during application of the milk sample to the sample pad of the lateral flow stick due to incorrect positioning of the needle during application of the milk sample to the sample pad is eliminated, or at least reduced.

Thanks to the described aspects, by determining the distance between the needle tip and the lateral flow stick when the needle is situated in the retracted position, biomarker values of milk samples of animals could successfully be determined by applying the milk sample to the lateral flow stick and run the lateral flow measurement.

Various states, e.g. related to reproduction of the animals, or various animal deceases or other anomalies could thus be determined with high reliability and without having to repeat failed lateral flow measurements due to incorrectly applied milk samples.

Other advantages and additional novel features will become apparent from the subsequent detailed description.

### FIGURES

Embodiments of the invention will now be described in further detail with reference to the accompanying figures, in which:

FIG. 1 illustrates an example of an arrangement for measuring a biomarker value of a milk sample of an animal, and a system.

FIG. 2A illustrates an example of a system and a moveable structure holding a needle of the milk dosing arrangement in a retracted position, at a distance from the lateral flow stick.

FIG. 2B illustrates an example of a system and a moveable structure holding the needle of the milk dosing arrangement in an application position, wherein the needle is situated when the milk sample is applied to the sample pad of the lateral flow stick.

FIG. 3A illustrates a side view of a lateral flow stick, according to an embodiment.

FIG. 3B illustrates a top view of a lateral flow stick, according to an embodiment.

### DETAILED DESCRIPTION

Embodiments of the invention described herein are defined as a system, which may be put into practice in the embodiments described below. These embodiments may, however, be exemplified and realised in many different forms and are not to be limited to the examples set forth herein; rather, these illustrative examples of embodiments are provided so that this disclosure will be thorough and complete.

Still other objects and features may become apparent from the following detailed description,considered in conjunction with the accompanying drawings. It is to be understood, however, that the drawings are designed solely for purposes of illustration and not as a definition of the limits of the herein disclosed embodiments, for which reference is to be made to the appended claims. Further, the drawings are not necessarily drawn to scale and, unless otherwise indicated, they are merely intended to conceptually illustrate the structures and procedures described herein.

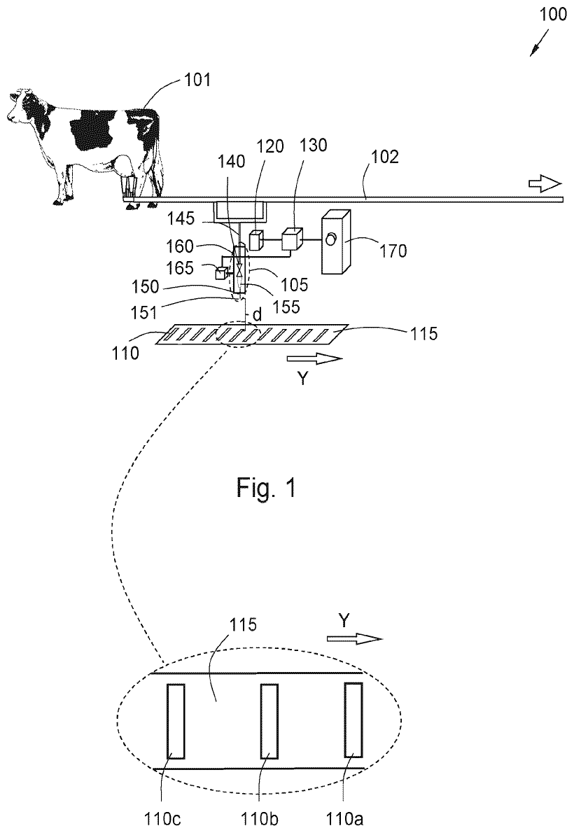

FIG. 1 illustrates a scenario with an animal 101 which may be comprised in a herd of dairy animals at a dairy farm and a system 100. The system 100 is arranged to monitor and control application of a milk sample extracted from an animal 101 to a sample pad of a lateral flow stick 110. The lateral flow stick 110 is arranged to indicate presence of a biomarker in a milk sample extracted from the animal 101.

“Animal” may be any arbitrary type of domesticated female milk producing mammal such as cow, goat, sheep, etc.

The lateral flow stick 110 may be individually arranged on a carrier 115 comprising a plurality of lateral flow sticks 110a, 110b, 110c. Each one of the individually arranged lateral flow sticks 110a, 110b, 110c on the carrier 115 may be arranged separated from each other.

The carrier 115 may be embodied e.g. as a carrier tape, or a blister package. In embodiments wherein the carrier 115 is embodied as a carrier tape, the carrier 115 may be comprised in a cassette, for a swift handling.

The carrier 115 may comprise a longitudinal extension in a Y-direction. An undisclosed driving arrangement may in some embodiments cooperate with the carrier 115 in order to move the carrier 115 in the Y-direction, thereby moving one lateral flow stick 110a, 110b, 110c at the time into a predetermined position in the Y-direction wherein application of the milk sample is enabled.

Each lateral flow stick 110 may be individually sealed by a foil covering the lateral flow stick 110 and the carrier 115. The foil may be made of transparent plastic/elastomere, for example. In these embodiments, the foil may be peeled off before the milk sample is applied to the lateral flow stick 110. In case the carrier 115 is embodied as a blister package, the seal cover of the blister package may be punctured before application of the milk sample to the lateral flow stick 110.

Milk of the animal 101 may be extracted and evacuated by a milking equipment that are part of a milking parlour via a milk line to a milk tank or similar milk storage.

During milking of the animal 101, or in close relation therewith, a milk sample is diverted from the milking equipment/milk line and provided to a milk dosing arrangement 105 comprised in the system 100. The milk dosing arrangement 105 may comprise for example a tube element 145 for attachment to an interface to the milking equipment/milk line, a pump configured to act on the tube element 145, for forwarding the milk sample. The milk dosing arrangement 105 may also comprise a valve 140, a mixing chamber 155 and a needle 150 for receiving the milk sample via the tube element 145.

In some embodiments, the milk sample may be mixed with a diluent in the mixing chamber 155, thereby forming a liquid sample comprising a mixture of milk and diluent.

The needle 150 of the milk dosing arrangement 105 may be aligned with the lateral flow stick 110, where after the milk sample/liquid sample may be applied via the needle 150 to the lateral flow stick 110 for a lateral flow measurement.

The system 100 comprises a moveable structure 160 arranged to hold the milk dosing arrangement 105 and to move the needle 150 of the milk dosing arrangement 105 between the application position and a retracted position. In the application position, the needle tip 151 of the needle 150 abuts the sample pad of the lateral flow stick 110. The needle 150 is arranged to apply the milk sample to the sample pad of the lateral flow stick 110 in the application position. In the retracted position, the needle tip 151 of the needle 150 is situated at a distance d from the lateral flow stick 110.

The system 100 also comprises a drive unit 165 such as a motor or engine. For example, the drive unit 165 may be a stepper motor in some embodiments. The drive unit 165 is arranged to act on the moveable structure 160, thereby causing the moveable structure 160 to move the needle 150 of the milk dosing arrangement 105 for a predetermined distance between the retracted position and the application position.

The system 100 comprises a controller 130. The controller 130 is communicatively connected to the drive unit 165. The controller 130 is configured to determine the distance d between a needle tip 151 of the needle 150 of the milk dosing arrangement 105 when the needle 150 is situated in the retracted position, and the lateral flow stick 110. The determined distance d is then compared with the predetermined distance.

The predetermined distance is the distance that the drive unit 165 cause the moveable structure 160 to move the needle 150 between the retracted position and the application position.

The controller 130 is also configured to compare the determined distance d with the predetermined distance; and, when the determined distance d is different from the predetermined distance, either update the predetermined distance to correspond with the determined distance d; or adjust the distance d between the needle tip 151 of the needle 150 of the milk dosing arrangement 105 when the needle 150 is situated in the retracted position, and the lateral flow stick 110, to correspond with the predetermined distance.

When the comparison results in detecting that the determined distance d is identical with the predetermined distance (possibly within a tolerance interval), it is possible to proceed, by performing the flow measurement on the lateral flow stick 110.

The adjustment of the distance d between the needle tip 151 of the needle 150 of the milk dosing arrangement 105 when the needle 150 is situated in the retracted position, and the lateral flow stick 110 may be made by adjustment of the position of the milk dosing arrangement 105, in relation to the lateral flow stick 110, i.e. the retracted position of the needle 150.

Thereafter, the controller 130 is enabled to run a lateral flow measurement. The needle tip 151 of the needle 150 of the milk dosing arrangement 105 is thereby positioned in the application position, so that the needle tip 151 abuts the sample pad of the lateral flow stick 110. The extracted milk sample; or a liquid sample comprising a mixture of milk and diluent may then be applied to the sample pad.

The lateral flow stick 110 may react on presence and/or amount of one or several biomarkers, e.g. by changing colours, colour tint or intensity of a colour/tint. A camera 120 comprised in the system 100 may capture an image. The camera 120 may be communicatively connected to the controller 130. The captured image of the lateral flow stick 110 may then be provided to and analysed by the controller 130, and based on the intensity of the colour, presence and/or quantity of the biomarker in the milk sample may be estimated.

The measured biomarker may be e.g. progesterone, glycoprotein, oestrogen and/or Gonadatropin-Releasing Hormones, or any other similar biomarker associated with reproduction of the animal 101, in different embodiments.

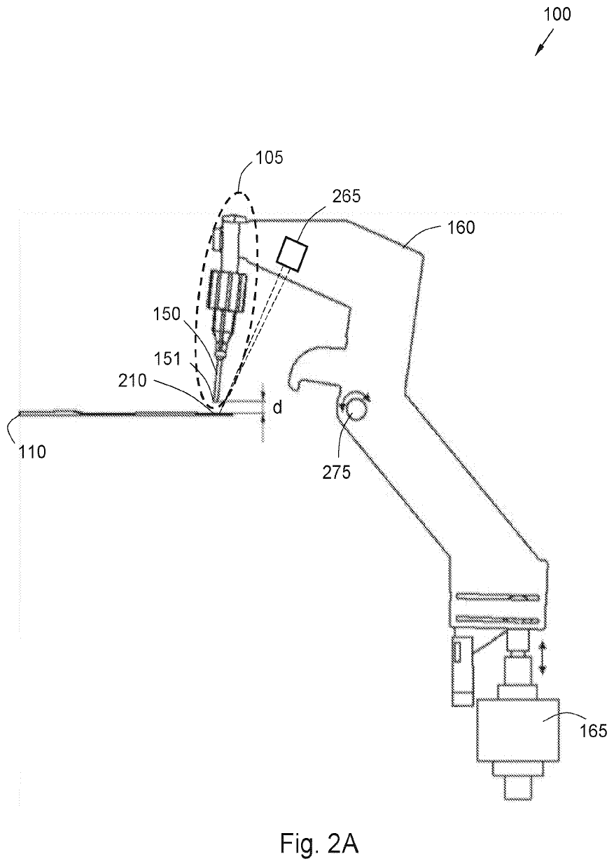

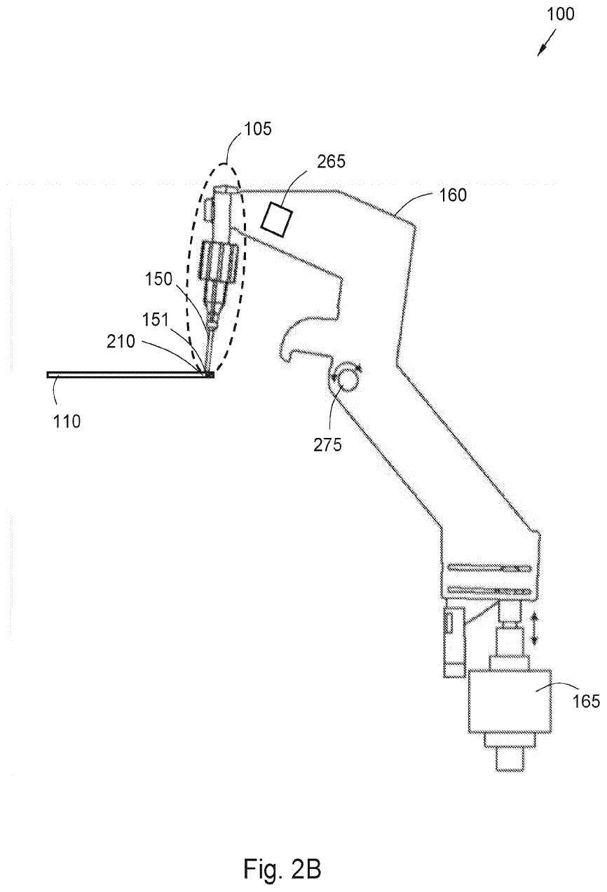

FIG. 2A schematically illustrates the device 100 comprising the moveable structure 160 which is holding the needle 150 of the milk dosing arrangement 105 in a retracted position, at a distance d from the lateral flow stick 110.

The drive unit 165 is arranged to act on the moveable structure 160, thereby causing the moveable structure 160 to move the needle 150 of the milk dosing arrangement 105, from the retracted position illustrated in FIG. 2A, into the application position illustrated in FIG. 2B. The drive unit 165 may comprise a linear motor or a stepper motor in different embodiments.

In the illustrated example, the drive unit 165 is turning the moveable structure 160 around an axle 275. This is however merely one possible solution out of plenty. The movement may be a linear movement, or a lever movement.

The controller 130 may be configured to determine the distance d between the needle tip 151 of the needle 150 of the milk dosing arrangement 105 when the needle 150 is situated in the retracted position, and the lateral flow stick 110. The lateral flow stick 110 may primarily be positioned in the predetermined position in the Y-direction wherein application of the milk sample is enabled via the needle 150.

The controller 130 may then be configured to set a speed of the drive unit 165 below a speed threshold limit.

The controller 130 may also be configured to cause the drive unit 165 to act on the moveable structure 160, thereby causing the moveable structure 160 to move the milk dosing arrangement 105 from the retracted position towards the carrier 115 and/or the lateral flow stick 110, for example by rotating the moveable structure 160 around the axle 275. In addition, the controller 130 may be configured to count each step moved by the drive unit 165 from the retracted position towards the lateral flow stick 110 until detecting that the needle tip 151 of the needle 150 abuts the sample pad 210 of the lateral flow stick 110.

The controller 130 may be configured to determining the distance d based on the counted number of steps moved by the drive unit 165 causing the moveable structure 160 to move the milk dosing arrangement 105 from the retracted position towards the the lateral flow stick 110 until the needle tip 151 of the needle 150 abuts the sample pad 210 of the lateral flow stick 110.

By counting the number of steps, and knowing the distance moved in each step, the distance d could be determined. The drive unit 165 may in this embodiment be a stepper motor.

In some embodiments, an estimated stick deflection distance may be reduced from the determined distance d. The estimated stick deflection distance may be a global constant which may be applied.

The controller 130 may be configured to detect that the needle tip 151 of the needle 150 abuts the sample pad 210 of the lateral flow stick 110 in several different ways in different embodiments.

In some embodiments, the controller 130 may be configured to detect that the needle tip 151 of the needle 150 abuts the sample pad 210 of the lateral flow stick 110 by detecting that the drive unit 165 is stalling.

For causing the drive unit 165 to stall when the needle tip 151 of the needle 150 abuts the sample pad 210, the controller 130 may be configured to set a motor power of the drive unit 165 below a power threshold limit. The power threshold limit may be set to a power level which assures that the drive unit 165 will stall when the needle tip 151 of the needle 150 abuts the sample pad 210.

In yet some embodiments wherein the system 100 comprises a camera 120, the controller 130 may be configured to detect that the needle tip 151 of the needle 150 abuts the sample pad 210 of the lateral flow stick 110 based on image analysis of an image captured by the camera 120.

The controller 130 may be configured to cause the camera 120 to capture at least one image of the lateral flow stick 110, for example a continuous sequence of images. The captured image or images may then be obtained from the camera 120, whereafter the controller 130 may be configured to perform an image analysis of the captured image/-s. Based on the outcome of the image analysis, the controller 130 may detect that the needle tip 151 of the needle 150 abuts the sample pad 210 of the lateral flow stick 110.

Based on analysis of the image, the controller 130 may detect light reflections created by the needle tip 151 when interacting with a foil covering the sample pad 210 of the lateral flow stick 110. The controller 130 may then be configured to detect that the needle tip 151 of the needle 150 abuts the sample pad 210 of the lateral flow stick 110 when detecting the light reflections by the foil.

The device 100 may comprise a sensor 265, communicatively connected to the controller 130. The sensor 265 may be arranged and directed to measure a distance that is directly or indirectly representative of the distance d, when the needle 150 of the milk dosing arrangement 105 is hold in the retracted position and the lateral flow stick 110 is positioned in the predetermined position in the Y-direction wherein application of the milk sample is enabled.

The sensor 265 may for example comprise a camera, a lidar, a radar, an ultrasound device, a time-of-flight camera, or similar device, in different embodiments.

The controller 130 may be configured to emit, via an emitting element of the sensor 265, an electromagnetic wave towards the lateral flow stick 110. A reflection of the emitted electromagnetic wave, reflected by the lateral flow stick 110 may then be received via a receiving element of the sensor 265.

The controller 130 may additionally be configured to measure a time period between the emission of the electromagnetic wave and the reception of the reflection of the emitted electromagnetic wave; and determine the distance d, directly or indirectly based on the measured time period, by knowing the speed of the emitted/reflected electromagnetic wave.

A direct measurement of the distance d between the needle tip 151 of the needle 150 of the milk dosing arrangement 105 when the needle 150 is situated in the retracted position, and the lateral flow stick 110 requires that the sensor 265 is situated at the position of the needle tip 151, directed towards the sample pad 210 of the lateral flow stick 110.

As this may be inconvenient, it may be an advantage to instead make an indirect measurement of the distance d between the needle tip 151 of the needle 150 of the milk dosing arrangement 105 when the needle 150 is situated in the retracted position.

The indirect measurement of the distance d may be made in several different ways by the sensor 265, by measuring a distance representative of the distance d, between the sensor 265 and the sample pad 210 of the lateral flow stick 110. The sensor 265 may then be mounted on the moveable structure 160 with a fixed relative distance between the sensor 265 and the needle tip 151 of the needle 150 of the milk dosing arrangement 105.

The controller 130 may be configured to determine the distance d at a regular time interval, for example every 24 hours, or some other appropriate or configurable time interval.

Alternatively, the controller 130 may be configured to determine the distance after a predetermined number of lateral flow measurements; i.e. the controller 130 may be configured to keep track on a number of lateral flow measurements made after the last distance determination of the distance d, and then repeat the distance determination after each n lateral flow measurements, where 1<n<1 000, for example about 400.

In embodiments wherein the carrier 115 comprising the plurality of lateral flow sticks 110a, 110b, 110c is replaceable, the controller 130 may be configured to detect the replacement of carriers 115. The controller 130 may then be configured to determine the distance d upon detection of the replacement of carriers 115.

The detection of the replacement of carriers 115 may be made by a sensor for detecting presence/absence/replacement of the carrier 115.

The carrier 115 may alternatively comprise a unique identifier, for example a code such as a QR code, bar code etc, which may be scanned by the camera 120 communicatively connected to the controller 130. Thereby, non-authorised carriers 115 and lateral flow sticks 110a, 110b, 110c may be detected. As these kinds of products typically is of low quality, a more intense distance determination scheme may be applied for non-authorised carriers 115 and lateral flow sticks 110a, 110b, 110c, for example every hour, every ten minutes, every minute, every ten seconds, before every lateral flow measurement, etc., for example.

When the code of an entrusted carrier 115 is scanned the controller 130 may be configured to determine the distance d relatively seldom, for example on the first lateral flow stick 110 on the carrier 115.

The device 100 may in some embodiments comprise a user interface 170, enabling an operator to trigger the distance determination of the distance d. The controller 130 may then be configured to determine the distance d when receiving a trigger signal from the operator via the user interface 170.

In yet some embodiments, the controller 130 may be configured to keep track on the successfulness or result of each performed lateral flow measurement, and when k number of unsuccessful lateral flow measurements have been made within a threshold period, the distance d may be determined. For example; 1<k<10 in some embodiments. The threshold period for triggering the distance determination may relate to a time period, such as 2 failed lateral flow measurements within 20 minutes. The threshold period for triggering the distance determination may alternatively relate to a number of lateral flow measurements, i.e. if 2 lateral flow measurements fail, out of the latest 10 lateral flow tests; or 2 failed lateral flow measurements in a row, the distance determination of the distance d may be triggered.

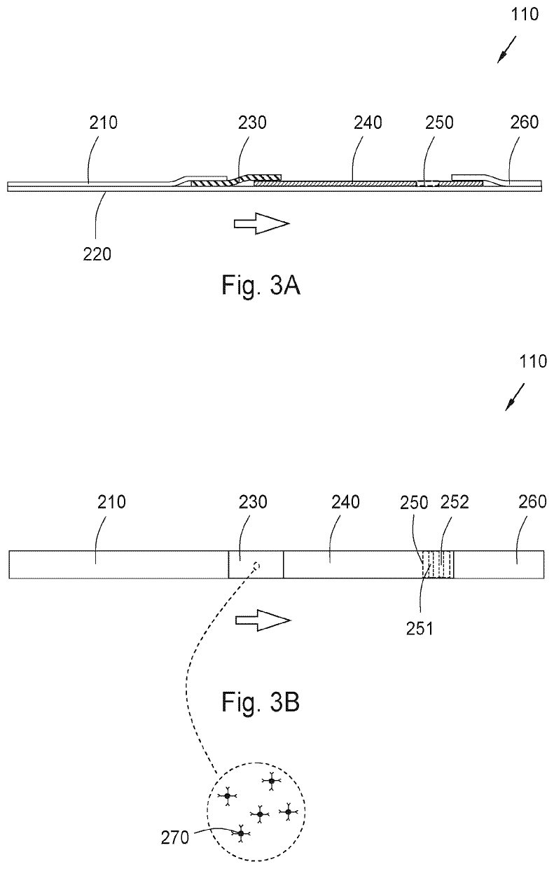

FIG. 3A illustrates a lateral flow stick 110 as regarded from a side view while FIG. 3B illustrates the lateral flow stick 110 as regarded from above.

The lateral flow stick 110 may comprise a back plate 220, which may be made of cardboard, paper or similar material. The back plate 220 may form a base on to which other components of the lateral flow stick 110 may be mounted.

The lateral flow stick 110 comprises a sample pad 210. The sample pad 210 is the part of the lateral flow stick 110 onto which the milk sample/liquid sample is applied by the needle 150 when the milk dosing arrangement 105 is situated in the application position. The sample pad 210 may comprise a porous structure for enabling capillary flow such as cellulose fibres and/or woven meshes.

The lateral flow stick 110 comprises a conjugate pad 230 comprising conjugate 270. The conjugate 270 may comprise antibody treated gold particles which are dispersed into the milk when the milk sample/liquid sample flows from the sample pad 210 through the conjugate pad 230.

The conjugate 270 will react with the biomarker to be measured, for example progesterone in the milk of the applied liquid sample.

The conjugate 270 may thus be embedded in the conjugate pad 230 which may comprise a glass-fibre section of the lateral flow stick 110. Alternatively, the conjugate pad 230 may comprise cellulose and/or surface modified polyester.

Also, the lateral flow stick 110 may comprise an indication zone 250, arranged to indicate the biomarker of the milk sample/liquid sample, based on the conjugate 270. The lateral flow stick 110 may furthermore comprise a porous membrane 240 for receiving a capillary flow of the milk sample/liquid sample from the conjugate pad 230, thereby forwarding conjugate 270 dispersed into the milk sample/liquid sample to the indication zone 250.

The porous membrane 240 may comprise for example a nitrocellulose membrane, cellulose, glass fibre, polyester, rayon, a polymer, glass fibre, woven fibres, non-woven fibres, a chromatographic gel membrane, diatomaceous earth, silica gel, silicium oxide, kieselguhr, or other filtration membranes in different embodiments. The porous membrane 240 may be designed to enhance capillary pumping speed of liquid through the lateral flow stick 110. The indication zone 250 of the porous membrane 240 may comprise a test line 251 and a control line 252.

The test line 251 may be treated with a biomarker reference such as e.g. a progesterone reference which binds the antibody treated gold particles of the milk sample/liquid sample and thereby brings the test line 251 to change colour tint when exposed for milk comprising a progesterone level lower than a threshold limit. Thus, the test line 251 may change colour tint into red/reddish when the milk has no or low progesterone level. If/when the animal 101 is in heat, the progesterone level is near zero in the milk sample. This colour change may then be detected by the camera 120 and may be reported to the farmer and/or stored in a database associated with an identity and/or a time reference of the animal 101. This may also trigger scheduling of insemination of the animal 101 (in case the biomarker is progesterone), or the scheduling of a veterinary check-up, for example (in case of another biomarker).

The control line 252 in the indication zone 250 of the porous membrane 240 may be treated with an antibody reference which binds antibody reference treated gold particles of the milk sample/liquid sample regardless of the progesterone level in the milk, and may thereby bring the control line 252 to change colour tint when exposed for milk comprising antibody reference treated gold particles, assuring a successful capillar flow of the milk/liquid through the porous membrane 240.

The lateral flow stick 110 may comprise an absorbent pad 260. The absorbent pad 260 may comprise an absorbent configured to absorb superfluous milk/liquid from the porous membrane 240.

The embodiments, or parts thereof, illustrated in FIG. 1, FIG. 2A, FIG. 2B, FIG. 3A, FIG. 3B may with advantage be combined with each other for achieving further benefits.

The terminology used in the description of the embodiments as illustrated in the accompanying drawings is not intended to be limiting of the described system 100. Various changes, substitutions and/or alterations may be made, without departing from invention embodiments as defined by the appended claims.

As used herein, the term “and/or” comprises any and all combinations of one or more of the associated listed items. The term “or” as used herein, is to be interpreted as a mathematical OR, i.e., as an inclusive disjunction; not as a mathematical exclusive OR (XOR), unless expressly stated otherwise. In addition, the singular forms “a”, “an” and “the” are to be interpreted as “at least one”, thus also possibly comprising a plurality of entities of the same kind, unless expressly stated otherwise. It will be further understood that the terms “includes”, “comprises”, “including” and/or “comprising”, specifies the presence of stated features, actions, integers, steps, operations, elements, and/or components, but do not preclude the presence or addition of one or more other features, actions, integers, steps, operations, elements, components, and/or groups thereof. A single unit such as e.g. a processor may fulfil the functions of several items recited in the claims. The mere fact that certain measures or features are recited in mutually different dependent claims, illustrated in different figures or discussed in conjunction with different embodiments does not indicate that a combination of these measures or features cannot be used to advantage. A computer program may be stored/distributed on a suitable medium, such as an optical storage medium or a solid-state medium supplied together with or as part of other hardware, but may also be distributed in other forms such as via Internet or other wired or wireless communication system.

1. A system configured to monitor and control application of a milk sample extracted from an animal to a sample pad of a lateral flow stick, wherein the lateral flow stick is configured to indicate presence of a biomarker in the milk sample extracted from an animal; wherein the system comprises:
a milk dosing arrangement, comprising
a tube element configured to obtain the milk sample; and
a needle configured to receive the milk sample via the tube element and apply the milk sample to the sample pad of the lateral flow stick when the milk dosing arrangement is held in an application position in which a needle tip abuts the sample pad; and
a moveable structure configured to hold the milk dosing arrangement and to move the needle of the milk dosing arrangement between
the application position, in which the needle tip abuts the sample pad of the lateral flow stick while applying the milk sample to the sample pad, and
a retracted position, in which the needle tip is situated at a distance (d) from the lateral flow stick;
a drive unit configured to act on the moveable structure, thereby causing the moveable structure to move the needle of the milk dosing arrangement a predetermined distance between the retracted position and the application position;
a controller, communicatively connected to the drive unit, wherein the controller is configured to
determine the distance (d) between the needle tip of the needle of the milk dosing arrangement when the needle is situated in the retracted position, and the lateral flow stick;
compare the determined distance (d) with the predetermined distance; and
either
update the predetermined distance to correspond with the determined distance (d) when the determined distance (d) is different from the predetermined distance; or
adjust the distance (d) between the needle tip of the needle of the milk dosing arrangement when the needle is situated in the retracted position, and the lateral flow stick, to correspond with the predetermined distance.

2. The system according to claim 1, wherein the needle tip of the needle of the milk dosing arrangement abuts a surface of the sample pad of the lateral flow stick when the needle of the milk dosing arrangement is situated in the application position.

3. The system according to claim 1, wherein the controller is configured to determine the distance (d) by:
setting a speed of the drive unit below a speed threshold limit;
causing the drive unit to act on the moveable structure, thereby causing the moveable structure to move the milk dosing arrangement from the retracted position towards the lateral flow stick;
counting each step moved by the drive unit until detecting that the needle tip of the needle abuts the sample pad of the lateral flow stick; and
determining the distance (d) based on the counted number of steps moved by the drive unit.

4. The system according to claim 3, wherein the controller is configured to determine the distance (d) by:
setting a motor power of the drive unit below a power threshold limit, and
detecting that the needle tip of the needle abuts the sample pad of the lateral flow stick by detecting that the drive unit is stalling.

5. The system according to claim 1, comprising a camera directed to capture an image of the lateral flow stick; and
wherein the controller is configured to directly or indirectly determine the distance (d) by:
causing the moveable structure to move the milk dosing arrangement from the retracted position towards the lateral flow stick;
causing the camera to capture at least one image of the lateral flow stick;
obtaining the captured image from the camera; and
detecting that the needle tip of the needle abuts the sample pad of the lateral flow stick, based on image analysis of the captured image.

6. The system according to claim 5, wherein the controller is configured to detect that the needle tip of the needle abuts the sample pad of the lateral flow stick by detecting light reflections created by the needle tip when the needle tip abuts a foil covering the sample pad of the lateral flow stick.

7. The system according to claim 1, comprising a sensor, communicatively connected to the controller, wherein the controller is configured to directly or indirectly determine the distance (d) by:
emitting, via an emitting element of the sensor, an electromagnetic wave towards the lateral flow stick;
receiving, via a receiving element of the sensor, a reflection of the emitted electromagnetic wave reflected by the lateral flow stick;
measuring a time period between the emission of the electromagnetic wave and the reception of the reflection of the electromagnetic wave; and
determine the distance (d), directly or indirectly, based on the measured time period.

8. The system according to claim 1, wherein the controller is configured to determine the distance (d) at a regular time interval.

9. The system according to claim 1, wherein the lateral flow stick is arranged on a carrier comprising a plurality of lateral flow sticks, which carrier is replaceable; wherein the controller is configured to detect a replacement of carriers; and wherein the controller is configured to determine the distance (d) upon detection of the replacement of carriers.

10. The system according to claim 1, comprising a user interface, enabling an operator to trigger the distance determination; and wherein the controller is configured to
determine the distance (d) when receiving a trigger signal from the operator via the user interface.

11. The system according to claim 2, wherein the controller is configured to determine the distance (d) by:
setting a speed of the drive unit below a speed threshold limit;
causing the drive unit to act on the moveable structure, thereby causing the moveable structure to move the milk dosing arrangement from the retracted position towards the lateral flow stick;
counting each step moved by the drive unit until detecting that the needle tip of the needle abuts the sample pad of the lateral flow stick; and
determining the distance (d) based on the counted number of steps moved by the drive unit.

12. The system according to claim 11, wherein the controller is configured to determine the distance (d) by:
setting a motor power of the drive unit below a power threshold limit, and
detecting that the needle tip of the needle abuts the sample pad of the lateral flow stick by detecting that the drive unit is stalling.

13. The system according to claim 3, comprising a camera directed to capture an image of the lateral flow stick; and
wherein the controller is configured to directly or indirectly determine the distance (d) by:
causing the moveable structure to move the milk dosing arrangement from the retracted position towards the lateral flow stick;
causing the camera to capture at least one image of the lateral flow stick;
obtaining the captured image from the camera; and
detecting that the needle tip of the needle abuts the sample pad of the lateral flow stick, based on image analysis of the captured image.

14. The system according to claim 2, comprising a sensor, communicatively connected to the controller, wherein the controller is configured to directly or indirectly determine the distance (d) by:
emitting, via an emitting element of the sensor, an electromagnetic wave towards the lateral flow stick;
receiving, via a receiving element of the sensor, a reflection of the emitted electromagnetic wave reflected by the lateral flow stick;
measuring a time period between the emission of the electromagnetic wave and the reception of the reflection of the electromagnetic wave; and
determine the distance (d), directly or indirectly, based on the measured time period.
